(19) United States
(12) Patent Application Publication
(10) Pub. No.: US 2008/0206329 A1
(43) Pub. Date: Aug. 28, 2008

(54) Modified Release Ciprofloxacin Compositions

(76) Inventors: Sanjay Verma, Haryana (IN); Subhash Pandurang Gore, Hyderabad (IN); Indu Bhushan, Haryana (IN); Vijay Dinanath Nasare, Maharashtra (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/720,583

(22) PCT Filed: Dec. 2, 2005
(86) PCT No.: PCT/US05/43656

§ 371 Date: May 31, 2007

(30) Foreign Application Priority Data
Dec. 3, 2004 (IN) 1308/CHE/2004

(51) Int. Cl. A61K 31/473; A61K 9/24; A61P 31/00

(52) U.S. Cl. 424/465; 514/312

(57) ABSTRACT

Controlled release tablets comprising ciprofloxacin, with hardness and friability of the said tablets ranges between 10 to 70 kiloponds and less than 2% respectively, the compositions releasing at least 80 percent of the total amount of the ciprofloxacin into a pH 1.2 aqueous dissolution medium within about 1 hour.

### INTRODUCTION TO THE INVENTION

The present invention relates to pharmaceutical compositions comprising ciprofloxacin or its pharmaceutically acceptable salts or combinations thereof, wherein the said compositions release at least a portion of the active ingredient in a controlled manner.

Ciprofloxacin belongs to the fluoroquinolone group of compounds and is chemically known as 1-cyclopropyl-6-fluoro- 1,4-dihydro-4-oxo-7-(1-piperazinyl ) -3-quinolinecarboxylic acid. It is structurally represented by Formula 1.

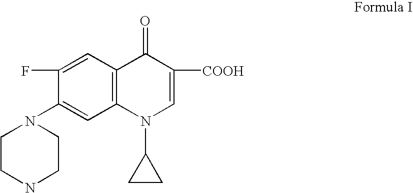

Ciprofloxacin is a broad-spectrum synthetic antimicrobial agent for oral administration. It demonstrates activity in vitro against a wide range of gram-negative and gram-positive organisms. It acts by inhibiting the enzymes topoisomerase II (DNA gyrase) and topoisomerase IV (both type II topoisomerases) which are required for bacterial DNA replication, transcription, repair and recombination. Its use as an antimicrobial agent has distinct advantages over the use of other antibiotics (e.g. penicillins, cephalosporins, aminoglycosides, sulphonamides and tetracyclines) in that ciprofloxacin does not induce tolerance or resistance in bacteria.

Ciprofloxacin is commercially available in pharmaceutical products in the United States and in other parts of the world for the treatment of bacterial infections. Ciprofloxacin hydrochloride is a monohydrated monohydrochloride salt of ciprofloxacin. It is available in the form of immediate release compositions under the brand name CIPRO® as 100 mg, 250 mg, 500 mg and 750 mg (ciprofloxacin equivalent) tablets.

Commercially, two strengths (500 mg and 1000 mg ciprofloxacin equivalent) of controlled release bilayer tablets are available. They utilize a combination of ciprofloxacin in the form of a hydrate having the formula C₁₇H₁₈FN₃O₃·3.5 H₂O (providing 212.6 mg or 425.2 mg ciprofloxacin on a dried basis) and a mixture of the monohydrate and sesquihydrate of ciprofloxacin hydrochloride (equivalent to 287.5 or 574.9 mg ciprofloxacin on a dried basis), and are sold using the brand name CIPRO XR®. Inactive ingredients used in the commercial products are crospovidone, hypromellose, magnesium stearate, polyethylene glycol, silica colloidal anhydrous, succinic acid and titanium dioxide. Active ingredient is present in immediate and controlled release forms as two different layers. Controlled release is achieved by an erosion-matrix principle using the water-swellable polymer hypromellose.

Controlled or modified release compositions have distinct advantages like enhanced patient compliance due to reduced frequency of dosing and reduced side effects due to reduced fluctuations in blood plasma levels of drug. Moreover, it is possible to maintain the blood plasma levels of the drug in the therapeutic range for a much longer period than what is achieved by using conventional compositions.

U.S. Patent Application Publication No. 2004/0024018 A discloses a sustained release matrix preparation of a quinolone active compound using a water-swellable hydrophilic polymer. The composition releases 80 percent of the active compound both in dissolution media of pH 1.2 comprising 0.1 N hydrochloric acid and in acetate buffer at pH 4.5, when tested by the USP XXIV paddle method at 50 revolutions per minute and 37° C., in the course of 1 to 4 hours.

It is known in the art that fluoroquinolones are mainly absorbed in the stomach and upper part of small intestine and the degree of absorption is very low from lower parts of intestine (S. Harder et al., “Ciprofloxacin absorption in different regions of the human gastrointestinal tract investigations with the HF-capsule,” British Journal of Clinical Pharmacology, Vol. 30(1), pages 35-39, 1990). Several attempts have been made in the art to develop pharmaceutical compositions that have increased the retention in the stomach or upper part of the intestines to cause maximum release of the active ingredient in this part of the gastrointestinal tract, thereby increasing the efficacy of therapy.

Some of the approaches that have been used to enhance retention of the dosage form in the stomach or upper part of the intestine include for example modification of the density of the preparation (European Patent Application No. 0265061 A); use of ballooning preparations (G. A. Agyilirah et al., “Evaluation of the gastric retention properties of a cross-linked polymer coated tablet versus those of a non-disintegrating tablet,” International Journal of Pharmaceutics, Vol. 75, pages 241-247, 1991); preparations having large spatial expansion (European Patent Application No. 0235718 A), and bioadhesive preparations (R. Khosla et al., “The effect of polycarbophil on the gastric emptying of pellets,” Journal of Pharmacy and Pharmacology, Vol. 39, page 47-49, 1987).

U.S. Pat. Nos. 6,261,601 and 6,960,356 describe a sustained release pharmaceutical composition in the form of a tablet or capsule that is retained in the stomach or upper part of the small intestine. The composition comprises a drug, a gas generating component, a swelling agent, a viscolyzing agent and optionally a gel-forming polymer.

U.S. Pat. No. 6,635,280 discloses formulations wherein the drug is incorporated into hydrophilic polymeric matrices that swell on imbibition of water to a size that is large enough to promote retention of the dosage form in the stomach during the fed mode.

U.S. Pat. No.4,777,033 discloses an oral sustained release pharmaceutical preparation comprising a cellulosic or acrylic polymer and active agent and optionally a foaming agent with enhanced residential persistence in the stomach.

The above-described compositions are complex to formulate and further complicate the challenges involved in the formulation of modified release compositions of high dose active ingredients like fluoroquinolones.

There is always a need for the development of newer modalities for delivering active compounds more effectively while being equivalent to commercially available compositions in terms of the in vitro and in vivo profiles. The availability of such alternative compositions in the market place provides the medical practitioner with additional tools for the more effective treatment of disease conditions.

Thus, a composition that is simple to make using conventional pharmaceutical processing means without using complex retention mechanisms; is stable and bioequivalent to a commercially available composition (such as for example CIPRO XR) and demonstrates maintenance of therapeutic blood levels of the pharmaceutical active for a prolonged duration enabling a once-a-day administration would fill a much-felt need in the marketplace.

### SUMMARY OF THE INVENTION

An aspect of the invention includes a tablet comprising:
a first layer comprising ciprofloxacin or a hydrate thereof, a salt of ciprofloxacin or a hydrate thereof, and a disintegrant; and
a second layer comprising ciprofloxacin or a hydrate thereof, a salt of ciprofloxacin or a hydrate thereof, and a water-insoluble release-retarding component.

Another aspect of the invention includes a tablet prepared by a method comprising compressing a solid mixture comprising a water-insoluble release-retarding component and at least one of:
ciprofloxacin, or a hydrate thereof; and
a salt of ciprofloxacin, or a hydrate thereof.

A further aspect of the invention includes a tablet comprising:
an immediate release layer comprising ciprofloxacin or a hydrate thereof, a hydrochloride salt of ciprofloxacin, or a hydrate thereof, and a disintegrant;
a modified release layer comprising ciprofloxacin or a hydrate thereof, a hydrochloride salt of ciprofloxacin, or a hydrate thereof, and a water-insoluble release-retarding component; and
optionally, a coating surrounding the tablet.

In an embodiment of the invention, at least about 80 percent of a total contained ciprofloxacin is released within about 1 hour, during immersion in an aqueous fluid having a pH about 1.2.

In an embodiment of the invention, at least about 80 percent of a total contained ciprofloxacin is released within about 1 hour, during immersion in an aqueous fluid having a pH about 1.2.

An embodiment of the tablet compositions comprises an immediate release portion and a modified release portion. In one embodiment, both of the portions are present in a single layered tablet. In another embodiment the portions are present in different layers of a multilayered tablet.

In an aspect, the hardness of the tablets of the invention ranges between 10 and 70 kiloponds and the tablets have a friability of less than about 2 percent.

### DETAILED DESCRIPTION

The present invention pertains to formulations for the delivery of ciprofloxacin in a controlled manner. More specifically, an embodiment of the invention relates to pharmaceutical compositions of ciprofloxacin in the form of tablets for once daily administration.

The active ingredient used in the compositions of the invention comprises ciprofloxacin or any of its pharmaceutically acceptable salts, esters, solvates, or hydrates, including combinations of any two or more thereof.

In an aspect, the invention relates to pharmaceutical compositions comprising ciprofloxacin that are characterized in that they release at least about 80 percent of the active ingredient(s) in a dissolution medium of pH 1.2 when tested using a USP XXVIII dissolution test apparatus (paddle type) at 50 revolutions per minute at 37° C., within about 1 hour. In a specific embodiment of the invention, at least about 90 percent of the total active ingredient is released into the pH 1.2 medium within about 1 hour.

In another aspect, the invention relates to pharmaceutical compositions that release at least about 60 percent of the total amount of the active ingredient(s) in a dissolution medium of pH 4.5 within about 1 hour when tested using a USP XXVIII dissolution test apparatus (paddle type) at 50 revolutions per minute at 37° C.

The pharmaceutical compositions of the invention comprise an immediate release (“IR”) portion and a modified release (“MR”) portion. These portions may be present in a single layer tablet or in a multi layer tablet.

Accordingly, in one embodiment of the invention, both of these portions are present in a single layer tablet. Thus, according to this embodiment of the invention one portion comprises an IR portion and other portions comprise MR portion. Such a composition can be prepared by incorporating together the different portions prepared separately such as for example IR granules and MR granules; or IR pellets and MR pellets.

In another embodiment these portions are present in different layers of a multi layer tablet. Thus, according to this embodiment of the invention one layer comprises an IR portion and another layer comprises an MR portion.

In one aspect, a bilayered tablet is provided comprising an IR layer which provides the IR portion of the invention and a further MR layer, which provides the MR portion of the dosage form.

The total amount of active ingredient present in the compositions of the invention varies from about 200 to 1500 mg, or about 500 to 1000 mg, of contained ciprofloxacin.

The ratio of ciprofloxacin to its salt present in the compositions of the invention varies from 20:1 to 1:20. In an embodiment, an IR portion comprises about 30-50 percent of the total active ingredient.

In one aspect of the invention, modified release is achieved by incorporating a water-insoluble release-retarding component in the said composition. The ratio of the active ingredient to water-insoluble release-retarding component varies from about 1:50 to 50:1, or about 1:20 to 20:1.

The water-insoluble release-retarding component can be a polymeric material such as, but not limited to: cellulosic polymers such as cellulose ethers like ethyl cellulose; cellulose esters such as cellulose acetate, cellulose propionate, cellulose acetate propionate, cellulose acetate butyrate, cellulose acetate phthalate and cellulose triacetate; acrylate and methacrylate polymers and copolymers, including, without limitation the methacrylic copolymers sold as EUDRAGIT™ L 100-55, L 30 D-55, L 100, and S 100, the methacrylate copolymers sold as EUDRAGIT™ NE 30 D and NE 40 D, and the ammonioalkyl methacrylate copolymers sold as EUDRAGIT™ RL 30 D, RL PO, and RS 100, all of the EUDRAGIT products being available from Röhm GmbH & Co. KG of Darmstadt, Germany; waxes such as beeswax, carnauba wax and microcrystalline wax; fatty alcohols such as cetostearyl alcohol, stearyl alcohol, cetyl alcohol, and myristyl alcohol; and fatty acid esters like glyceryl monostearate, glycerol monooleate, acetylated monoglycerides, tristearin, tripalmitin, glyceryl palmitostearate, and glyceryl behenate; and hydrogenated castor oil. Combinations of water-insoluble release-retarding components are also useful in the invention.

In one specific embodiment of the invention, ethyl cellulose is used as a water-insoluble release-retarding component.

In another specific embodiment, an ammonioalkyl methacrylate copolymer is used as a water-insoluble release-retarding component.

In an aspect of the invention, ciprofloxacin is used in the said composition.

In another aspect of the invention, ciprofloxacin hydrochloride is used in the said composition.

In yet another aspect, a combination of ciprofloxacin hydrochloride and ciprofloxacin is used to prepare the compositions of the present invention.

Wet granulation, dry granulation, direct compression or other processes known in the art can be used to prepare the said IR and MR portions of the compositions. The said compositions optionally comprise pharmaceutical excipients such as, but not limited to, diluents, binders, disintegrants, lubricants, glidants, film formers, plasticizers and colourants.

Common diluents useful in the present invention include, but are not limited to, microcrystalline cellulose, silicified microcrystalline cellulose, microfine cellulose, lactose, starch, pregelatinized starch, calcium carbonate, calcium sulfate, sugar, dextrates, dextrin, dextrose, mannitol, sorbitol, dibasic calcium phosphate dihydrate, tribasic calcium phosphate, kaolin, magnesium carbonate, magnesium oxide, maltodextrin, polymethacrylates, and mixtures thereof.

Binders useful in the present invention include, but are not limited to, starches, microcrystalline cellulose, methylcellulose, cellulose ethers, sodium carboxymethylcellulose, ethylcellulose, dextrose, lactose, sucrose, sorbitol, mannitol, polyethylene glycol, polyvinylpyrrolidone (PVP), pectins, gelatin, polyacrylamides, polyvinyloxoazolidone, polyvinylalcohols, and mixtures thereof.

Disintegrants useful in the present invention include, but are not limited to, partially hydrolyzed polyvinyl alcohol, cellulose ethers, starch and gelatin.

Lubricants useful in the present invention include, but are not limited to, colloidal silicon dioxide, stearic acid, magnesium stearate, calcium stearate, talc, hydrogenated castor oil, sucrose esters of fatty acid, microcrystalline wax, yellow beeswax and white beeswax.

Glidants useful in the present invention include, but are not limited to, colloidal silicon dioxide, stearic acid, magnesium stearate, calcium stearate and talc.

Acidifying agents useful in the present invention include, but are not limited to, ascorbic acid, citric acid, fumaric acid, glutamic acid, malic acid, maleic acid, succinic acid, tartaric acid, and the like. These agents are used alone or in combination in IR portion or modified release portion or both.

IR and MR portions are blended separately or together, with or without disintegrants or lubricants or glidants and compressed using the tablet tooling either as single layer tablets or bilayer tablets on a rotary tablet presses.

The hardness of the tablets of the invention varies from about 10 to 70 kiloponds (about 90 to 700 newtons) or about 15 to 50 kiloponds (about 140 to 500 newtons). The hardness may be measured by any conventional hardness tester such as for example a Strong Cobb, Monsanto, VanKel (Varian), Erweka, Pfizer, Schleuniger, or Pharma hardness tester.

The friability of the tablet of the invention typically is less than about 2 percent. The friability can be measured using a friability tester as described in the United States Pharmacopoeia, Edition 28, page 2745.

The tablets of the said composition can optionally be coated with film forming agents known in the art. Such film forming agents include, but are not limited to, different grades of hydroxypropyl methylcellulose, hydroxypropyl cellulose, methyl cellulose, hydroxyethyl cellulose and mixtures thereof. The coating optionally comprises other pharmaceutically acceptable additives such as but not limited to solvents, plasticizers, colorants, emulsifying agents and solubilizers. Commercially available coating formulations such as those being sold using the OPADRY™ and OPAGLOS™ brands are also useful in the present invention. Other pharmaceutically acceptable additives known in the art for the coating of pharmaceutical compositions are also within the scope of this invention without limitation and can be appropriately selected by a person skilled in the art of manufacture of coated pharmaceutical solid oral dosage forms.

Coating techniques useful for the coating of the tablets of this invention include without limitation spray coating, dip coating, fluidized bed coating, or other processes known in the art that can accomplish coating of the said composition.

In an embodiment of the invention, the tablets have one or more of the following pharmacokinetic parameters, determined by analyzing plasma ciprofloxacin concentrations following oral administration of a dose containing 1000 mg of ciprofloxacin to a human subject:
AUC_{0-t about} 11,000-19,000 ng·h/ml;
AUC_{0-∞} about 12,000-20,000 ng·h/ml;
Cₘₐₓ about 2,000-3,300 ng/ml; and
Tₘₐₓ about 2-4.5 hours.

In another embodiment of the invention, the tablets have one or more of the following pharmacokinetic parameters, determined by analyzing plasma ciprofloxacin concentrations following oral administration of a dose containing 1000 mg of ciprofloxacin to a human subject:
AUC₀₋ₜ about 13,000-17,000 ng·h/ml;
AUC_{0-∞} about 14,000-18,000 ng·h/ml;
Cₘₐₓ about 2,000-3,000 ng/ml; and
Tₘₐₓ about 2.5-4.5 hours.

These pharmacokinetic parameters are defined in accordance with accepted regulatory terms: Cₘₐₓ is the maximum plasma concentration of the active ingredient that is achieved after dosing; Tₘₐₓ is the elapsed time after dosing, until achieving Cₘₐₓ; AUC₀₋ₜ is the area under the plasma concentration-time curve, beginning at the time of dosing and ending at last time point at which the plasma concentration of active ingredient can be measured; and AUC_{0-∞} is the area under the plasma concentration-time curve, beginning at the time of dosing and ending at a time when the extrapolated active ingredient concentration would be expected to be zero.

The controlled release compositions of ciprofloxacin of the present invention are useful in the treatment of medical conditions requiring administration of ciprofloxacin or its pharmaceutically acceptable salts thereof. Such conditions include treatment of urinary tract infections, cystitis, prostatitis, skin, lung, airway, bone, joint infections, and other infections in human beings and other mammals.

The following examples further illustrate certain aspects and embodiments of the invention in greater detail and are not intended to limit the scope of the invention in any manner.

### EXAMPLE 1

Bilayer tablet using ciprofloxacin and its hydrochloride salt, 1000 mg ciprofloxacin equivalent

| | |
|---|---|
| Component | mg/Tablet |

IR Portion

| | |
|---|---|
| Ciprofloxacin hydrochloride | 368.12 (316.15 mg |
| | ciprofloxacin |
| | equivalent) |
| Ciprofloxacin | 34.02 |
| Crospovidone* | 48 |
| Magnesium stearate | 5 |
| Colloidal silicone dioxide | 2.4 |
| Water | q.s. |

MR Portion

| | |
|---|---|
| Ciprofloxacin hydrochloride | 301.18 (258.65 mg |
| | ciprofloxacin |
| | equivalent) |
| Ciprofloxacin | 391.18 |
| Succinic acid | 124 |
| Ethyl cellulose (7 cps) | 78 |
| Magnesium stearate | 15 |
| Colloidal silicon dioxide | 5 |
| Isopropanol | q.s. |

Film Coating Composition

| | |
|---|---|
| OPADRY White** | 27.438 |
| Water | q.s. |

*Crospovidone is a synthetic cross-linked homopolymer of N-vinyl-2-pyrrolidone.
**OPADRY ™ White is a formulated film coating material sold by Colorcon, West Point, Pennsylvania U.S.A., containing hydroxypropyl methylcellulose 2910/hypromellose 6 cps, titanium dioxide and talc.

Manufacturing process:

I. IR portion
1. Ciprofloxacin hydrochloride, ciprofloxacin and crospovidone were passed through an ASTM 20 mesh sieve and mixed in a rapid mixer granulator for two minutes using a slow speed of the impeller.
2. The blend of step 1 was granulated with water.
3. The granules of step 2 were dried in a fluidized bed dryer at 60° C. until a loss on drying (LOD) below 4% was achieved when measured at 105° C.
4. The dried granules were mixed with magnesium stearate and colloidal silicon dioxide in a double cone blender for 10 minutes.

II. MR portion
1. Ciprofloxacin hydrochloride and ciprofloxacin were passed through an ASTM 20 mesh sieve.
2. Succinic acid was milled in a comminuting mill and passed through an ASTM 80 mesh sieve.
3. Components of step 1 and 2 were mixed for two minutes in a rapid mixer granulator at low speed.
4. Ethyl cellulose was dissolved in isopropyl alcohol.
5. The mixture of step 3 was granulated using the ethyl cellulose solution of step 4.
6. The granules were dried in a fluidized bed dryer at 60° C. until LOD was less than 2% when measured at 105° C.

7. The dried granules were mixed with magnesium stearate and colloidal silicon dioxide in a double cone blender for 10 minutes.

III. Compression

The granules obtained in I and 11 above were compressed into bilayer tablets using capsule shaped punches (23 mm×9.5 mm) on a double rotary tablet compression machine.

IV. Coating
1. Opadry white was dispersed in water.
2. The bilayer tablets of III were coated with the Opadry dispersion using a perforated coating pan.

### EXAMPLE 2

In-vitro drug release from tablets made according to Example 1, at pH 1.2*
Dissolution apparatus: USP XXIV dissolution test Apparatus 2 (paddle type)
Dissolution medium: 0.1 N hydrochloric acid, pH 1.2
Volume of dissolution medium: 900 ml
Stirring speed: 50 rpm
Hardness of the tablets: 29-40 kiloponds as measured using a VanKel hardness tester (sold by Varian, Inc., Palo Alto, Calif. U.S.A.)
Friability of the tablets: 0.1% measured using a USP friability tester.

United States Pharmacopeia 24, Drug Release Test 724, pages 1944-1951.

Drug Released (%)

| Time (min) | Example 1 | CIPRO ® XR (1000 mg) |
|---|---|---|
| 30 | 45 | 53.58 |
| 60 | 95 | 71.67 |
| 120 | 101 | 95.83 |

### EXAMPLE 3

In-vitro drug release from tablets made according to Example 1, at pH 4.5
Dissolution apparatus: USP XXIV dissolution Apparatus 2 (paddle type)
Dissolution medium: Acetate buffer (pH 4.5)
Volume of dissolution medium: 900 ml
Stirring speed: 50 rpm

Drug Released (%)

| Time (min) | Example 1 | CIPRO ® XR (1000 mg) |
|---|---|---|
| 30 | 41 | 39 |
| 60 | 63 | 63 |
| 90 | 89 | 82 |
| 120 | 95 | 94 |

### EXAMPLE 4

Bioequivalence study using the composition of Example 1

An open label, balanced, randomized two-treatment, two-period, two-sequence single dose cross-over bioequivalence study was performed in the fed state that included fourteen subjects, with at least 7 days washout period between each drug administration. The results of this study, from analyses of plasma ciprofloxacin concentrations after administering 1000 mg of ciprofloxacin, are shown in the following table:

| | Reference | Composition of | | Coefficient |
|---|---|---|---|---|
| Parameter | (R)* | Example 1 (T) | T/R (%) | of variation |
| AUC₀₋ₜ | 14807 | 16251 | 109.7 | 9.7 |
| (ng · h/ml) | | | | |
| AUC_{0-∞} | 15738 | 16864 | 107.1 | 9.7 |
| (ng · h/ml) | | | | |
| Cₘₐₓ (ng/ml) | 2606 | 2664 | 102.2 | 12.1 |
| Tₘₐₓ (hours) | 3.25 | 4 | — | — |

*Reference used was CIPRO ® XR 1000 mg tablets

### EXAMPLE 5

Uncoated bilayered tablet for the controlled release of ciprofloxacin 500 mg

| | |
|---|---|
| Component | mg/Tablet |

IR Portion

| | |
|---|---|
| Ciprofloxacin hydrochloride | 195.26 |
| Ciprofloxacin | 25.87 |
| Crospovidone* | 24 |
| Magnesium stearate | 2.5 |
| Colloidal silicon dioxide | 1.2 |
| Water | q.s. |

MR Portion

| | |
|---|---|
| Ciprofloxacin hydrochloride | 159.76 |
| Ciprofloxacin | 189.73 |
| Succinic acid | 60 |
| Ethyl cellulose | 50 |
| Magnesium stearate | 7.5 |
| Colloidal silicon dioxide | 2.5 |
| Isopropanol | q.s. |

The composition was prepared in a manner similar to Example 1 except that the bilayered tablets obtained in III were not coated subsequently.

### EXAMPLE 6

Uncoated bilayered tablet for the controlled release of ciprofloxacin 1000 mg

| | |
|---|---|
| Component | mg/Tablet |

IR Portion

| | |
|---|---|
| Ciprofloxacin hydrochloride | 390.52 |
| Ciprofloxacin | 51.75 |
| Crospovidone | 48 |
| Magnesium stearate | 5 |
| Colloidal silicon dioxide | 2.4 |
| Water | q.s. |

MR Portion

| | |
|---|---|
| Ciprofloxacin hydrochloride | 319.51 |
| Ciprofloxacin | 379.47 |
| Succinic acid | 120 |
| Ethyl cellulose | 100 |
| Magnesium stearate | 15 |
| Colloidal silicon dioxide | 5 |
| Isopropanol | q.s. |

The composition was prepared in a manner similar to Example 1 except that the bilayered tablets obtained in III were not coated subsequently.

### EXAMPLE 7

Bilayer tablet comprising ciprofloxacin hydrochloride alone

| | |
|---|---|
| Component | mg/Tablet |

IR Portion

| | |
|---|---|
| Ciprofloxacin hydrochloride | 400 |
| Crospovidone | 47 |
| Magnesium stearate | 5 |
| Colloidal silicon dioxide | 2.4 |
| Water | q.s. |

MR Portion

| | |
|---|---|
| Ciprofloxacin hydrochloride | 600 |
| Succinic acid | 120.3 |
| Ethyl cellulose | 100 |
| Magnesium stearate | 7.5 |
| Colloidal silicon dioxide | 2.5 |
| Isopropanol | q.s. |

The composition was prepared in a manner similar to Example 1 except that the bilayered tablets were prepared using ciprofloxacin hydrochloride instead of a combination of ciprofloxacin hydrochloride and ciprofloxacin. The tablets obtained in III were not subsequently coated.

### EXAMPLE 8

Bilayer tablet with ciprofloxacin alone

| | |
|---|---|
| Component | mg/Tablet |

IR Portion

| | |
|---|---|
| Ciprofloxacin | 450 |
| Crospovidone | 45 |
| Magnesium stearate | 5 |
| Colloidal silicon dioxide | 2.4 |
| Water | q.s. |

MR Portion

| | |
|---|---|
| Ciprofloxacin | 550 |
| Succinic acid | 120.3 |
| Ethyl cellulose | 100 |
| Magnesium stearate | 7.5 |
| Colloidal silicon dioxide | 2.5 |
| Isopropanol | q.s. |

The composition was prepared in a manner similar to Example 1 except that the bilayered tablets were prepared using ciprofloxacin hydrochloride instead of a combination of ciprofloxacin hydrochloride and ciprofloxacin. The tablets obtained in Ill were not subsequently coated.

### EXAMPLE 9

Single layer tablet with ciprofloxacin and its hydrochloride salt

| Component | mg/Tablet |
|---|---|
| Ciprofloxacin hydrochloride | 343.31 |
| Ciprofloxacin | 215.39 |
| Ethyl cellulose | 75 |
| Citric acid | 65 |
| Magnesium stearate | 10 |
| Colloidal silicon dioxide | 3.3 |
| Isopropanol | q.s. |
| Water | q.s. |

Manufacturing process:
1. The first four ingredients were separately passed through an ASTM 40 mesh sieve and blended in a rapid mixer granulator for two minutes at a slow speed of the impeller.
2. The blend of step 1 was granulated using a mixture of isopropyl alcohol and water.
3. The granules of step 2 were dried in a fluidized bed dryer at 60° C. until a loss on drying below 4% was achieved when measured at 105° C.
4. The dried granules were mixed with magnesium stearate and colloidal silicon dioxide in a double cone blender for 10 minutes.
5. The blend of step 4 was compressed into tablets using 17.8×9.4 mm punches.

### EXAMPLE 10

Bilayer tablet

| | |
|---|---|
| Component | mg/Tablet |

IR Portion

| | |
|---|---|
| Ciprofloxacin hydrochloride | 368.12 |
| Ciprofloxacin | 34.02 |
| Crospovidone | 48 |
| Magnesium stearate | 5 |
| Colloidal silicone dioxide | 2.4 |
| Water | q.s. |

MR Portion

| | |
|---|---|
| Ciprofloxacin hydrochloride | 301.18 |
| Ciprofloxacin | 391.18 |
| Succinic acid | 124 |
| EUDRAGIT ™ RL PO* | 91.43 |
| Magnesium stearate | 15 |
| Colloidal silicon dioxide | 5 |
| Isopropanol | q.s. |

Film Coating Composition

| | |
|---|---|
| Opadry White | 27.438 |
| Water | q.s. |

*Ammonioalkyl methacrylate copolymer, from Röhm GmbH & Co. KG, Darmstadt, Germany.

The composition was prepared in a manner similar to Example 1, except that in step 4 EUDRAGIT RL PO was dissolved in isopropyl alcohol and added in place of ethyl cellulose.

1. A tablet comprising:
a first layer comprising ciprofloxacin or a hydrate thereof, a salt of ciprofloxacin or a hydrate thereof, and a disintegrant; and
a second layer comprising ciprofloxacin or a hydrate thereof, a salt of ciprofloxacin or a hydrate thereof, and a water-insoluble release-retarding component.

2. The tablet of claim 1, wherein a salt of ciprofloxacin comprises ciprofloxacin hydrochloride.

3. The tablet of claim 1, wherein a water-insoluble release-retarding component comprises ethyl cellulose.

4. The tablet of claim 1, wherein a water-insoluble release-retarding component comprises an ammonioalkyl methacrylate copolymer.

5. The tablet of claim 1, having a hardness about 10 to about 70 kiloponds.

6. The tablet of claim 1, having a hardness about 15 to about 50 kiloponds.

7. The tablet of claim 1, wherein at least about 80 percent of a total contained ciprofloxacin is released within about 1 hour during immersion in an aqueous fluid having a pH about 1.2.

8. The tablet of claim 1, wherein at least about 90 percent of a total contained ciprofloxacin is released within about 1 hour during immersion in an aqueous fluid having a pH about 1.2.

9. The tablet of claim 1, wherein a second layer is formed from granules prepared using a solution comprising a water-insoluble release-retarding component.

10. A tablet prepared by a method comprising compressing a solid mixture comprising a water-insoluble release-retarding component and at least one of:
ciprofloxacin, or a hydrate thereof; and
a salt of ciprofloxacin, or a hydrate thereof.

11. The tablet of claim 10, wherein a salt of ciprofloxacin comprises ciprofloxacin hydrochloride.

12. The tablet of claim 10, wherein a water-insoluble release-retarding component comprises ethyl cellulose.

13. The tablet of claim 10, wherein a water-insoluble release-retarding component comprises an ammonioalkyl methacrylate copolymer.

14. The tablet of claim 10, wherein a solid mixture comprises granules formed using a solution comprising a water-insoluble release-retarding component.

15. A tablet comprising:
an immediate release layer comprising ciprofloxacin or a hydrate thereof, a hydrochloride salt of ciprofloxacin, or a hydrate thereof, and a disintegrant;
a modified release layer comprising ciprofloxacin or a hydrate thereof, a hydrochloride salt of ciprofloxacin, or a hydrate thereof, and a water-insoluble release-retarding component; and
optionally, a coating surrounding the tablet.

16. The tablet of claim 15, wherein a modified release layer is formed from granules prepared using a solution comprising a water-insoluble release-retarding component.

17. The tablet of claim 15, wherein a water-insoluble release-retarding component comprises ethyl cellulose.

18. The tablet of 15, wherein at least about 80 percent of a total contained ciprofloxacin is released within about 1 hour during immersion in an aqueous fluid having a pH about 1.2.

19. The tablet of claim 15, wherein at least about 90 percent of a total contained ciprofloxacin is released within about 1 hour during immersion in an aqueous fluid having a pH about 1.2.

20. The tablet of claim 15 that produces, upon oral administration of a single dose containing 1000 mg of ciprofloxacin to a human, a ciprofloxacin plasma AUC₀₋ₜ about 11,000 to about 19,000 ng·h/ml.

21. The tablet of claim 15 that produces, upon oral administration of a single dose containing 1000 mg of ciprofloxacin to a human, a ciprofloxacin plasma AUC₀₋ₜ about 13,000 to about 17,000 ng·h/ml.

22. The tablet of claim 15, wherein at least about 80 percent of a total contained ciprofloxacin is released within about 1 hour during immersion in an aqueous fluid having a pH about 1.2, and that produces, upon oral administration of a single dose containing 1000 mg of ciprofloxacin to a human, a ciprofloxacin plasma AUC₀₋ₜ about 11,000 to about 19,000 ng·h/ml.

23. The tablet of claim 15 that produces, upon oral administration of a single dose containing 1000 mg of ciprofloxacin to a human, one or more of the pharmacokinetic parameters:
AUC₀₋ₜ about 11,000-19,000 ng·h/ml;
AUC_{0-∞} about 12,000-20,000 ng·h/ml;
Cₘₐₓ about 2,000-3,300 ng/ml; and
Tₘₐₓ about 2-4.5 hours; determined by analyzing ciprofloxacin concentration in plasma.

24. The tablet of claim 15 that produces, upon oral administration of a single dose containing 1000 mg of ciprofloxacin to a human, one or more of the pharmacokinetic parameters:
AUC₀₋ₜ about 13,000-17,000 ng·h/ml;
AUC_{0-∞} about 14,000-18,000 ng·h/ml;
Cₘₐₓ about 2,000-3,000 ng/ml; and
Tₘₐₓ about 2.5-4.5 hours; determined by analyzing ciprofloxacin concentration in plasma.

25. The tablet of claim 16, wherein at least about 80 percent of a total contained ciprofloxacin is released within about 1 hour during immersion in an aqueous fluid having a pH about 1.2.

26. The tablet of claim 17, wherein at least about 80 percent of a total contained ciprofloxacin is released within about 1 hour during immersion in an aqueous fluid having a pH about 1.2.

27. The tablet of claim 16, wherein at least about 90 percent of a total contained ciprofloxacin is released within about 1 hour during immersion in an aqueous fluid having a pH about 1.2.

28. The tablet of claim 17, wherein at least about 90 percent of a total contained ciprofloxacin is released within about 1 hour during immersion in an aqueous fluid having a pH about 1.2.

29. The tablet of any of claim 16 that produces, upon oral administration of a single dose containing 1000 mg of ciprofloxacin to a human, a ciprofloxacin plasma AUC₀₋ₜ about 11,000 to about 19,000 ng·h/ml.

30. The tablet of any of claim 17 that produces, upon oral administration of a single dose containing 1000 mg of ciprofloxacin to a human, a ciprofloxacin plasma AUC₀₋ₜ about 11,000 to about 19,000 ng·h/ml.

31. The tablet of claim 16 that produces, upon oral administration of a single dose containing 1000 mg of ciprofloxacin to a human, a ciprofloxacin plasma AUC₀₋ₜ about 13,000 to about 17,000 ng·h/ml.

32. The tablet of claim 17 that produces, upon oral administration of a single dose containing 1000 mg of ciprofloxacin to a human, a ciprofloxacin plasma AUC₀₋ₜ about 13,000 to about 17,000 ng·h/ml.

33. The tablet of claim 16, wherein at least about 80 percent of a total contained ciprofloxacin is released within about 1 hour during immersion in an aqueous fluid having a pH about 1.2, and that produces, upon oral administration of a single dose containing 1000 mg of ciprofloxacin to a human, a ciprofloxacin plasma AUC₀₋ₜ about 11,000 to about 19,000 ng·h/ml.

34. The tablet of claim 17, wherein at least about 80 percent of a total contained ciprofloxacin is released within about 1 hour during immersion in an aqueous fluid having a pH about 1.2, and that produces, upon oral administration of a single dose containing 1000 mg of ciprofloxacin to a human, a ciprofloxacin plasma AUC₀₋ₜ about 11,000 to about 19,000 ng·h/ml.

35. The tablet of claim 16 that produces, upon oral administration of a single dose containing 1000 mg of ciprofloxacin to a human, one or more of the pharmacokinetic parameters:
AUC₀₋ₜ about 11,000-19,000 ng·h/ml;
AUC_{0-∞} about 12,000-20,000 ng·h/ml;
Cₘₐₓ about 2,000-3,300 ng/ml; and
Tₘₐₓ about 2-4.5 hours; determined by analyzing ciprofloxacin concentration in plasma.

36. The tablet of claim 17 that produces, upon oral administration of a single dose containing 1000 mg of ciprofloxacin to a human, one or more of the pharmacokinetic parameters:
AUC₀₋ₜ about 11,000-19,000 ng·h/ml;
AUC_{0-∞} about 12,000-20,000 ng·h/ml;
Cₘₐₓ about 2,000-3,300 ng/ml; and
Tₘₐₓ about 2-4.5 hours; determined by analyzing ciprofloxacin concentration in plasma.

37. The tablet of claim 16 that produces, upon oral administration of a single dose containing 1000 mg of ciprofloxacin to a human, one or more of the pharmacokinetic parameters:
AUC₀₋ₜ about 13,000-17,000 ng·h/ml;
AUC_{0-∞} about 14,000-18,000 ng·h/ml;
Cₘₐₓ about 2,000-3,000 ng/ml; and
Tₘₐₓ about 2.5-4.5 hours; determined by analyzing ciprofloxacin concentration in plasma.

38. The tablet of claim 17 that produces, upon oral administration of a single dose containing 1000 mg of ciprofloxacin to a human, one or more of the pharmacokinetic parameters:
AUC₀₋ₜ about 13,000-17,000 ng·h/mI;
AUC_{0-∞} about 14,000-18,000 ng·h/ml;
Cₘₐₓ about 2,000-3,000 ng/ml; and
Tₘₐₓ about 2.5-4.5 hours; determined by analyzing ciprofloxacin concentration in plasma.
